(19) United States
(12) Patent Application Publication
(10) Pub. No.: US 2004/0247527 A1
(43) Pub. Date: Dec. 9, 2004

(54) Multifunctional photodynamic agents for treating of disease

(75) Inventors: Charles W. Spangler, Livingston, MT (US); Aleksander Rebane, Bozeman, MT (US)

Correspondence Address: Robin M. Silva, Esq., Dorsey & Whitney LLP, Intellectual Property Department, Four Embarcadero Center, Suite 3400, San Francisco, CA, 94111-4187, US

(73) Assignee: MPA Technologies, Inc.

(21) Appl. No.: 10/805,683

(22) Filed: Mar. 10, 2004

Related U.S. Application Data
(60) Provisional application No. 60/453,618, filed on Mar. 10, 2003.

(51) Int. Cl. A61K 49/00; C07D 487/22

(52) U.S. Cl. 424/9.61; 540/145; 530/391.1

(57) ABSTRACT

The present invention is directed to methods and compositions comprising multifunctional (usually bi- or tri-functional) agents that incorporate a targeting moiety, a photo dynamic therapy (PDT) moiety (either one or two photon), and an optional imaging agent (such as a chromophore, contrast agent, etc.).

### CROSS-REFERENCE TO RELATED APPLICATIONS

This application is a Continuation-in-Part Application claiming benefit of priority to U.S. Ser. No. 60/453,618, filed Mar. 10, 2003 and is expressly incorporated by reference in its entirety.

### FIELD OF THE INVENTION

The present invention is directed to methods and compositions comprising multifunctional (usually bi- or tri-functional) agents that incorporate a targeting moiety, a photo dynamic therapy (PDT) moiety (either one or two photon), and an optional imaging agent (such as a chromophore, contrast agent, etc.).

### BACKGROUND OF THE INVENTION

Over the past five years there has been an ongoing renaissance in the development of new imaging and treatment technologies for the early detection of cancerous tumors. Despite the efforts of legions of cancer researchers over the past few decades, cancer is still the second leading cause of death among Americans, exceeded only by heart disease. There will be more than 1.2 million new cases of cancer diagnosed in the U.S. alone in 2002. Of these, ca. 70% will be solid cancerous tumors that should be amenable to early detection by a variety of in vivo imaging technologies currently under active development. From a patient and healthcare cost perspective, noninvasive imaging technologies that do not require an overnight hospital stay are highly desirable, particularly if it becomes possible to eliminate follow-up, confirmatory surgical biopsies. If these new imaging technologies could also be coupled with a noninvasive treatment procedure in the same patient session, then a complete outpatient imaging/detection/treatment protocol could be designed that might replace the current diagnosis/surgery/chemotherapy/ionizing radiation therapy protocol that has been the standard of treatment for the past twenty years.

The World Health Organization has estimated that more than 1.2 million new cases of cancer will be diagnosed in 2002, and of these, the American Cancer Society estimates that ca. 203,500 will be invasive breast cancer (defined as Stages I-IV), leading to an estimated 40,000 deaths see reference 9, incorporated by reference. Early detection is critical to long-term successful breast cancer and enhanced survival rates, as illustrated in Table 1.

TABLE 1
5-Year survival rates for breast cancer patients

| Breast Cancer | 5-Year Relative |
|---|---|
| Stage | Survival Rate |
| 0 | 100% |
| I | 98 |
| II a | 88 |
| II b | 76 |
| III a | 56 |
| III b | 49 |
| IV | 16 |

Clearly, early detection is a major factor in survival rates, but 20-40% of breast cancers go undetected at the yearly routine mammograms screening stage, where the smallest tumor that can be detected is 0.5-1.0 cm. In addition, many women experience severe discomfort during a typical mammogram procedure, particularly from breast compression. While it is generally regarded that woman over the age of 40 should undergo annual screening, it has been estimated by the American Cancer Society that only 62% of women in this category actually had a mammogram during the past year. These estimates point to the need for new screening procedures that both detect cancer at an earlier stage, and eliminate the discomfort factor.

Most recently, the efficacy of routine mammography has been revisited in the popular press and media (e.g., Time Magazine, 2/4 and 2/18 issues, 2002), primarily based on work published recently calling into question the interpretation of years of data supporting mammography as a primary screening device for healthy women. Most breast cancers originate in the milk ducts, and eventually develop into an early stage cancer referred to as a ductal carcinoma in situ, or DCIS, a pre-invasive localized stage that has not yet progressed outside of the breast ducts. Traditional mammography can be plagued by false positives at this stage, which can dictate that a follow-up mammogram be carried out, with possible additional confirmation sought by needle biopsy. It is not unusual for women to exhibit multiple scarring from unnecessary biopsies and surgeries, although the smallest DCISs can sometimes be treated by excision alone, with the caveat that wide cancer-free margins around the excised tumor are necessary to circumvent follow-on chemotherapy and ionizing radiation treatments.

The development of alternative imaging and treatment protocols that are truly noninvasive and could, (a) detect DCISs in their earliest possible stage, and (b) allow for non-surgical treatment, would be most welcome by women at high risk.

Over the past ten years there have been a number of excellent reviews that discuss both the potential and problems associated with in vivo optical imaging of cancerous tumors, particularly for the imaging of breast cancer (see references 17-20, incorporated by reference). Several of these authors have pointed out the need for high affinity vector molecules targeted against tumor-associated markers, and the need to increase uptake of a contrast agent into the tumor versus surrounding healthy tissue. Monoclonal antibodies have been used in this regard (see references 21-23, incorporated by reference), and Becker and coworkers have recently shown that macromolecules such as transferrin and human serum albumin conjugates with indotricarbocyanine (ITCC) are effective contrast agents for the optical imaging of tumors (see reference 24, incorporated by reference). Several disadvantages of using antibodies or other large molecules are that they can be taken up by the liver, elicit adverse immunological reactions in humans, and can have very long residence times in the blood system. In addition, large molecules may not be able to easily penetrate deep into the tumor due to positive interior pressure.

A possible solution to the problems associated with large molecule-contrast agent conjugates is to use small molecules, such as small peptides, to direct the contrast agents to the targeted tumors. A large number of tumors have been shown to overexpress receptors for somatostatin (SST) and other peptides (see references 25-28, incorporated by reference), and receptor scintigraphy for gastroentero-pancreatic tumors is in routine clinical use. Tumor targeting and imaging utilizing a somatostatin analog-fluorescent conjugate is an attractive alternative for optical imaging of cancerous tumors. Becker, et al., have recently proposed receptor-targeted optical imaging of tumors based on NIR fluorescent ligands attached to octreoate, a stable somatostatin small peptide analog (see reference 29, incorporated by reference). In their approach, indocyanine dyes such as indodicarbocyanine (IDCC) and indotricarbocyanine (ITCC) were coupled to octreoate utilizing Fmoc solid phase peptide synthesis methodology. A linear analog, a modified octreoate with methionine replacing the cysteines, was utilized as a control. The ITCC-octreoate accumulated in mice xenografts bearing an RIN38/SSTR2 tumor. The fluorescence contrast between the tumor and normal tissue immediately increased (ca. 1 minute), and from 3-24 hours the flouresence intensity of the tumor was more than threefold higher than surrounding normal tissue. Thus the small peptide somatostatin analogs were able to accumulate in the tumor quickly, and companion experiments also showed that they cleared from the system quickly after 24 hours. The linear octreoate-ITCC conjugate did not accumulate in the tumor, which underlines the necessity of careful matching of the somatostatin conjugate to the overexpressed tumor receptor sites. High affinity SST receptors are also overexpressed in the majority of breast carcinomas (see reference 30, incorporated by reference). Reubi and coworkers (see references 31-32, incorporated by reference) have examined a large number of human tumor types with respect to the expression and localization of somatostatin receptors SSTR1, SSTR2, and SSTR3 messenger RNAs and SS autoradiography and mRNA in situ hybridization. SS receptors were found in all breast tumors, with SSTR2 dominating, and were shown to have high affinity for octreoate. SST2 is the human somatostatin receptor subtype with the highest affinity for commercially available synthetic analogs.

Hawrysz and Sevick-Muraca (see reference 33, incorporated by reference) have pointed out in their excellent review that with the development of new imaging agents whose absorption/fluoresence is red-shifted towards the NIR, deeper tissue penetration can be achieved.

Recent work has been directed to a new design approach to porphyrins with greatly enhanced two-photon cross-sections, and we have proved in principle that these new porphyrin structural motifs are capable of extremely efficient 2-photon induced in vitro generation of singlet oxygen, the agent generally accepted as being the cause of cancer cell apoptosis in one-photon photo-dynamic therapy; see references 1-4, incorporated by reference.

However, there is a need to for treatments and modalities that are truly non-invasive and that can accomplish imaging and treatment in a single outpatient session.

### SUMMARY OF THE INVENTION

In accordance with the objects outlined above, the present invention provides bi- and trifunctional agents, comprising a targeting moiety and at least a photo dynamic therapy (PDT) moiety, preferably a two photon PDT moiety (2PM). The agents optionally comprise an imaging agent, preferably an optical imaging agent such as a chromophore or fluorophore, with one-photon chromophores being particularly preferred. In addition, the agents optionally but usually comprise a linker, to allow covalent attachment of the components of the agents.

The present invention further provides methods of detecting and/or treating disease, most notably cancer, by the activation of the PDT moiety using light at the appropriate wavelength to activate the moiety. The methods can also be combined with other imaging modalities.

### DESCRIPTION OF THE FIGURES

FIG. 1 is a schematic of the energy levels for porhyrin photosensitizer (solid bars) and molecular oxygen (open bars). S₀(g), S₁(u), Sᵢ(g), and T₁ represent, respectively, ground, first singlet, Ah excited singlet, and lowest triplet states of the photosensitizer. The symbols in the parenthesis denote gerarde (g) and unegerade (u) symmetry of the corresponding states ³Σ\overline{g} and ¹Δ_{g} denote the ground and the first excited singlet states of molecular oxygen.

FIG. 2 is a depiction of a preferred bifunctional agent.

FIG. 3 is a depiction of some preferred bifunctional and trifunctional agents.

FIG. 4 depicts some preferred trifunctional components.

FIG. 5 depicts some preferred TPA PDT chromophores for attachment to the multifunctional agents.

### DETAILED DESCRIPTION OF THE INVENTION

The present invention is directed to multifunctional compounds that combine several facets of the imaging and treatment of tumors (or other diseases) into a single reagent that can used in a one or more outpatient sessions for the detection and treatment of the disease. In general, subcutaneous cancerous tumors are a good candidate due to the ability suitable wavelength requirements of two photon agents, as described below, with the understanding that the use of endoscopes can allow the detection and treatment of other types of tumors, including solid tumors.

The multifunctional agents can be bifunctional or trifunctional. Bifunctional agents include a targeting moiety linked, generally via a linker, to a two-photon photodynamic moiety (2PM). The targeting moiety allows the covalently associated 2PM to accumulate rapidly in the tissue of choice (e.g. the tumor) and not to any substantial degree in surrounding and/or healthy tissue. The 2PM is capable of being activated by two-photon absorption of NIR photons to initiate the death of the diseased cells, e.g. cancer cells.

Trifunctional agents contain a targeting moiety, an imaging agent and a PDT moiety (PM), which can be either a single photon PM or a 2PM. As described below, the imaging agent allows the rapid three-dimensional imaging of the diseased tissue (e.g. cancerous tumors). When an imaging agent is combined with a 2PM, the resulting agent can be activated by NIR pulsed laser irradiation in the tissue transparency window (800-1000 nm). This covalently bound ensemble thus incorporates dual functionality: it can be employed in an imaging mode at low laser power, activating only the one-photon imaging agent, or it can operate as a photodynamic therapy reagent by changing the laser focus and increasing the power. The two-photon process will only become activated at the focus of the laser beam at the tumor site, and will have little or no effect on surrounding healthy tissue. Two-photon photodynamic therapy has long been a goal of several academic researchers and small companies (see references 5-7, incorporated by reference), but progress in this approach to cancer treatment has been limited due to the extremely small two-photon cross-sections of naturally occurring porphyrins, or commercial reagents such as Photofrin (see reference 8, incorporated by reference). However, the recent development of synthetic porphyrin materials with greatly enhanced two-photon cross-sections now make true two-photon PDT a practical alternative to one-photon PDT. See US Publication No. 2003/0105070, hereby incorporated by reference in its entirety, particularly with respect to the 2PM structures.

The combination of traditional imaging/contrast chromphores and two-photon PDT chromophores in the same reagent gives a new approach to an outpatient screening and detection system that also incorporates the potential for immediate photodynamic therapy treatment of any potentially cancerous growths discovered in the imaging process. The direct treatment of cancer then becomes directly linked to routine (e.g., yearly) screening, offering the twin advantages of early detection and nonsurgical outpatient treatment. This approach also offers the potential as a separate adjunct to other imaging technologies currently in use, or under development, such as traditional and digital mammography. Thus these agents can serve as a powerful new paradigm for in vivo detection and treatment of early-stage cancerous tumors. The advantages of this approach is exemplified in the following discussion of how it might be used in the treatment of early stage breast cancer tumors, however, the treatment protocol can be used for cancerous tissues, including solid tumors, at any stage of development.

Accordingly, the present invention provides multifunctional agents as described herein. In a preferred embodiment, the agents are trifunctional or triad compositions comprising three different components: a targeting moiety, an imaging moiety and a PDT moiety. As outlined below, linker moieties that serve to covalently attach the three components are frequently used.

By “targeting moiety” or grammatical equivalents herein is meant a functional group which serves to target or direct the complex to a particular location, cell type, diseased tissue, or association. In general, the targeting moiety is directed against a target molecule. As will be appreciated by those in the art, the agents of the invention are generally injected intraveneously; thus preferred targeting moieties are those that allow concentration of the agents in a particular localization accessible to the vascular system, although direct injection into body cavities (such as the spinal cord, interstitial spaces of the joints, etc.) is also possible. In a preferred embodiment, the agent is partitioned to the location in a non-1:1 ratio. Thus, for example, antibodies, cell surface receptor ligands and hormones, lipids, sugars and dextrans, alcohols, bile acids, fatty acids, amino acids, proteins (including peptides) and nucleic acids may all be attached to localize or target the contrast agent to a particular site.

In a preferred embodiment, the targeting moiety allows targeting of the agents of the invention to a particular tissue or the surface of a cell. That is, in a preferred embodiment the agents of the invention need not be taken up into the cytoplasm of a cell to be useful. In addition, preferred targeting moieties are against cancer targets. “Cancer targets” are those that are preferentially expressed or synthesized in cancer cells, tissues and/or tumors. For example, suitable cancer target substances include, but are not limited to, enzymes and proteins (including peptides) such as cell surface receptors; nucleic acids; lipids and phospholipids. Preferred embodiments utilize cancer targets that are on the surface of solid tumors, such as the somatostatin (SST) receptor outlined above, the HER2 receptor, etc., as outlined below.

In a preferred embodiment, the targeting moiety is a protein. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration. As discussed below, when the protein is used as a targeting moiety, it may be desirable to utilize protein analogs to retard in vivo degradation by proteases.

In a preferred embodiment, the protein is a binding partner (ligand) of a cell surface receptor, particularly those associated with disease, such as cancer cell surface receptors that are either specific to the cancerous tissue or differentially expressed. It is important to note that while high specificity of the targeting moiety to the disease tissue is preferred, since the irradiation can be targeted, it is not necessary that complete specificity (e.g. no binding to healthy tissue) exists. Cell surface ligands and/or analogs and derivatives, including fragments, are preferred, as are enzyme substrates or inhibitors, particularly of cell surface bound enzymes.

In a preferred embodiment, the targeting moiety is all or a portion (e.g. a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF-α and TGF-β), EPO receptor (EPO), TPO receptor (TPO), ciliary neurotrophic factor receptor, prolactin receptor, and T-cell receptors. In particular, hormone ligands are preferred. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathryroid hormone, thyroid-stimulating hormone (TSH), vasopressin, enkephalins, seratonin, estradiol, progesterone, testosterone, cortisone, and glucocorticoids and the hormones listed above. Receptor ligands include ligands that bind to receptors such as cell surface receptors, which include hormones, lipids, proteins, glycoproteins, signal transducers, growth factors, cytokines, and others. Somatostatin and transferring are particularly preferred.

Thus, in a preferred embodiment, the protein is a peptide, particularly those that are known to bind to cancer-specific cell surface receptors. Somatostatin, transferrin, and functional derivatives thereof are particularly preferred. Furthermore, chemotactic peptides have been used to image tissue injury and inflammation, particularly by bacterial infection; see WO 97/14443, hereby expressly incorporated by reference in its entirety. In addition, there are a wide variety of enzymes implicated in cancer, with associated peptides that will bind these enzymes, either as substrates or inhibitors, that can correspondingly be used as targeting moieties.

Cathepsin B is implicated in tumor invasion and progression. Cathepsin B secretion from cells may be induced by an acidic pH of the medium, although it is functional at physiological pH. It is a protein in the extracellular matrix (ECM) degrading protease cascade and undergoes autodegradation in the absence of a substrate. Cathepsin B has been implicated in breast, cervix, ovary, stomach, lung, brain, colorectal, prostate and thyroid tumors. It is active at the local invasive stage, with stage 1V tumors exhibiting significantly higher concentrations than lower staged tumors. It has been shown to be active at the tumor cell surface, at focal adhesions and invadopodia where the tumor cells contact the basal membrane and ECM. It degrades the ECM, both intracellularly and extracellularly, and includes laminin, fibronectin and collagen IV as its natural substrates. Suitable additional and synthetic substrates for use in the invention include, but are not limited to, edestin, gelatin, azo-casein, Benzyloxycarbonylarginylarginine 4-methylcoumarin-7-ylamine (Z-Arg-Arg-NH-Mec); trypsinogen; Benzyloxycarbonylphenylarginine 4-methylcoumarin-7-ylamine (Z-Phe-Arg-NH-Mec); N-a-benzyloxycarbonyl-L-arginyl-L-arginine 2-naphthylamide (Z-Arg-Arg-NNap); setfin A; Benzyloxycarbonylarginylarginine p-nitroanilide (Z-Arg-Arg-p-NA); oxidized β chain of insulin; Benzyloxycarbonylphenylarginine p-nitroanilide (Z-Phe-Arg-p-NA); a-N-benzoyl-L-arginine amide (BM); a-N-benzoyl-L-arginine ethyl ester (BAEE); a-N-benzoyl-D,L-arginine 2-napthylamide (BANA); a-N-benzoyl-D,L-arginine p-nitroanilide (BAPA); a-N-benzoyl-L-lysine amide (BLA); a-N-benzyloxycarbonyl glycine p-nitrophenyl ester (CGN); and a-N-benzyloxycarbonyl-L-lysine p-nitrophenyl ester (CLN). See Buck et al., Biochem. J. 282 (Pt 1), 273-278 (1992); Moin et al., Biochem. J. 285 (Pt 2), 427-434 (1992); Hasnain et al., Biol. Chem. Hoppe Seyler 373, 413-418 (1992); Willenbrock et al., Biochem. J. 227, 521-528 (1985); Otto, K. in Tissue Proteinases (Barrett, A. J. and Dingle, J. T., eds.) p. 1, North-Holland, Amsterdam; Bajkowski et al. Anal. Biochem 68, 119-127 (1975) and references therein, all of which are expressly incorporated by reference, and all of which can be used as targeting moieties.

In addition, there are a wide variety of known inhibitors, such as cystatin C, 1-(L-transepoxysuccinylleucylamino)-4-guanidinobutane (also called E-64 or (N-[N-(L-3-trans-carboxyoxiran-2carbonyl)-L-leucyl]-agmatine). See Yan et al., (1998) Biol. Chem. 379:113; Keppler et al., (1994); Biochem. Soc. Trans. 22:43; Hughes et al., PNAS USA 95:12410 (1998); Abdollahi et al., J. Soc. Gynecol. Invest. 6:32 (1999), Varughese et al., Biochemistry 31, 5172-5176 (1992); Hasnain et al, J. Biol. Chem. 267, 4713-4721 (1992), all of which are expressly incorporated by reference, and all of which can be used as targeting moieties.

In a preferred embodiment, the targeting moiety is a substrate or inhibitor for for cathepsin D. Cathepsin D is a 48 kDa aspartyl endoprotease with a classic Asp-Thr-Gly active site. Similar to a variety of other cathepsins, it is made as a 52 kDa precursor, procathepsin D. It is ubiquitously distributed in lysosomes. Cathepsin D has been implicated in breast, renal cell, ovary and melanoma cancers, and appears to be involved in the growth of micrometastases into clinical metastases. In tumor cells, cathepsin D is secreted into the surrounding medium resulting in delivery to the plasma membrane. Similar to cathepsin B, cathepsin D is part of the ECM degrading cascade of proteases. In addition, cathepsin D requires an acidic pH (4.5-5.0) for optimal activity. See Rochefort et al., APMIS 107:86 (1999); Xing et al., Mol. Endo. 12(9): 1310 (1998); Yazlovitskaya et al., Proc. Am. Assoc. Cancer Res. 37:#3553 519 (1996); all of which are expressly incorporated by reference, and all of which can be used as targeting moieties.

Known cathepsin D substrates and inhibitors include, but are not limited to, substrates: gp-120 and naphthazarin (5,8-dihydroxyl-1,4-naphthoquinone) and inhibitors: pepstatine and equistatin. See Ollinger, Archives of Biochemistry & Biophysics. 373(2):346-51, 2000; E I Messaoudi et al., Journal of Virology. 74(2):1004-7, 2000; Bessodes et al., Biochemical Pharmacology, 58(2):329-33, 1999; and Lenarcic et al., Journal of Biological Chemistry. 274(2):563-6, 1999, all of which are expressly incorporated by reference, and all of which can be used as targeting moieties.

In a preferred embodiment, the targeting moiety is a substrate or inhibitor for cathepsin K. Cathepsin K is also an elastolytic cysteine protease, and is considered to be the most potent mammalian elastase, and also has collagenolytic activity. Cathepsin K is considered unique among mammalian proteinases in that its collagenolytic activity does not depend on the destabilization of the triple helix of collagen in contrast to other cysteine proteases and that it cleaves native molecules at more sites than does interstitial collagenase. Thus, cathepsin K can degrade completely the insoluble collagen of adult cortical bone in the absence of other proteases. It is highly expressed in osteoclasts. It plays an important role in bone resorption and is essential for normal bone growth and remodeling. It has been implicated in osteoporosis, pycnodysotosis, bone cancer as well as breast cancer. It is interesting to note that, breast cancer commonly metastasizes to bone, and cathepsin K was initially identified as related to breast cancer by its presence in breast cancer cells that had spread to and invaded bone. Its substrates include, but are not limited to, elastin and collagen, and its inhibitors include, but are not limited to, Cbz-Gly-Arg-AMC; Cbz-Arg-Arg-AMC; Cbz-Gly-Gly-Arg-AMC; Cbz-Ala-Lys-Arg-AMC; Cbz-Ala-Arg-Arg-AMC; Cbz-d-Phe-Arg-AMC; Boc-Leu-Gly-Arg-AMC; H-Gly-Arg-AMC; H-Ala-Arg-AMC; Cbz-Leu-Leu-Leu-AMC; Cbz-Leu-Leu-AMC; Cbz-Phe-Gly-AMC; Cbz-Gly-Gly-Leu-AMC; Suc-Ala-Ala-Val-AMC; Cbz-Gly-Ala-Met-AMC; E-64; Leupeptin (Ac-Leu-Leu-Arg-CHO); N-acetyl-Leu-Leu-methional; Ac-Leu Leu-Met-CHO; Ac-Leu-Val-Lys-CHO; Ac-Leu-Leu-Nle-CHO; Cbz-Lys-Leu-Leu-CHO; Cbz-Leu-LeuLeu-CHO; Cbz-Arg-Leu-Leu-CHO; Series of 1,3-bis(acylamino)-2-propanones; series of 1,3 diamino ketones; and a series of 1,5-diacylcarbohydrazides. Suitable cathepsin K substrates include, but are not limited to, Cbz-Leu-Arg-AMC; Cbz-Val-Arg-AMC; Cbz-Phe-Arg-AMC; Cbz-Leu-Leu-Arg-AMC; Tos Gly-Pro-Arg-AMC; Bz-; Phe-Val-Arg-AMC; H-Pro-Phe-Arg-AMC; Cbz-Val-Val-Arg-AMC; Boc-Val-ProArg-AMC; Cbz-Glu-Arg-AMC; Bz-Arg-AMC; Ac-Phe-Arg-AMC; Boc-Val-Leu-Lys-AMC; Suc-Leu-TyrAMC; Boc-Ala-Gly-Pro-Arg-AMC; Cbz-Gly-Pro-Arg-AMC; Z-Leu-Arg-4-methoxy-b-naphthylamide (where Cbz=benzyloxycarbonyl and AMC=aminomethylcoumarin); diaminopropanones, diacylhydrazine and cystatin C. See Bossard, M. J. et al., J. Biol. Chem. 271, 12517-12524 (1996); Aibe, K. et al., Biol. Pharm. Bull. 19,1026-1031 (1996); Votta, B. J. et al. J. Bone Miner. Res. 12, 13961406 (1997); Yamshita, D. S. et al. J. Am. Chem. Soc. 119,11351-11352 (1997); DesJarlais, R. L. et al. J. Am. Chem. Soc. 120, 9114-9115 (1998); Marquis, R. W. et al. J. Med. Chem. 41, 3563-3567 (1998); Thompson et al., J. Med. Chem. 41, 3923-3927 (1998); Thompson et al., Bioorg. Med. Chem. 7, 599605 (1999); Kamiya, T. et al. J. Biochem. (Tokyo) 123, 752-759 (1998), Shi et al, J. Clin. Invest. 104:1191 (1999); and Sukhova et al., J. Clin. Invest. 102:576 (1998), all of which are expressly incorporated by reference, and all of which can be used as targeting moieties.

In a preferred embodiment, the targeting moiety is a substrate or inhibitor for β-glucuronidase. β-glucuronidase has been implicated in breast, colorectal and small cell lung carcinomas. β-glucuronidasehydrolyzes the glucuronide bond at the non-reducing termini of glycosamino carbohydrates. A variety of substrates are cleaved by β-glucuronidase, including, but not limited to, phenolphthalein glucuronide, 5-bromo-4-chloro-3-indoly-β-glucuronide, etc. The concentration of β-glucuronidase has been shown to be low in well differentiated cell lines and high in poorly differentiated (carcinoma) cell lines. In addition, β-glucuronidase activity has been detected in stromal cells which penetrate tumors and in necrotic areas of solid tumors, where it is liberated by host inflammatory components, mainly by monocytes and granulocytes. The enzyme from cancerous tissue has been shown to be phosphorylated on carbohydrates and proteins at serine and threonine positions. β-glucuronidase is an exoglycosidase that is a homotetramer of 332 kDa. It is transported to the lysosome by the man-6-P/IGFII receptor where it is released by the acidic medium. See Feng et al., Chin. Med. J. 112(9):854 (1999); Fujita et a I., GANN 75:598 (19840; Minton et al., Br. Canc. Res. Treat. 8:217 (1986); Pearson et al., Cancer 64:911 (1989); Bosslet et al., Canc. Res. 58:1195 (1998); Jain et al., Nat. Struc. Bio. 3:375 (1998); Ono et al., J. Biol. Chem. 263:5884 (1988), all of which are expressly incorporated herein by reference.

In a preferred embodiment, the targeting moiety is a substrate or inhibitor for heparanase. Heparanase has been implicated in breast, bladder, prostate, colon, hepatocellular and cervix carcinomas, metastatic melanoma, neuroblastoma, mesothelioma and endothelioma. It is an endoglucuronidase (sometimes referred to as a proteoglycanase) of 50 kDA, with an inactive 65 kDa form. It is secreted by highly metastatic tumor cells, activated T-lymphocytes, mast cells, platelets and neutrophils, and appears to be involved in invasion and metastasis of tumor cells. The expression of heparanase has been correlated with the metastatic potential of lymphoma, fibrosarcoma and melanoma cell lines, and has been detected in the urine of tumor-bearing patients. Its substate is heparan sulfate proteoglycans which are essential in the self-assembly and insolubility of the extracellular matrix. There are a variety of known inhibitors, including heparin and other anti-coagulant molecules of polysulfated polysaccharides such as phosphomanno-pentose sulfate. See Vlodasvsky et al., Nature Med. 5:793 (1999); Hulett et al., Nature Med. 5:803 (1999), both of which are incorporated by reference, and all of which can be used as targeting moieties.

In a preferred embodiment, the targeting moiety is a substrate or inhibitor for hepsin. Hepsin has been implicated in ovarian cancer, and appears to be involved in tumor invasion and metastasis by allowing implantation and invasion of neighboring cells. It is a serine protease with a classic catalytic triad (ser-his-asn), and may activate matrix metalloproteinases (MMP). It degrades the ECM through peptide bond cleavage, and is found extracellularly. See Tantimoto et al., Proc. Am. Assoc. Cancer Res. 38:(#2765):413 (1997).

In a preferred embodiment, the targeting moiety is a substrate or inhibitor for a matrix metalloproteinase (MMP), of which a variety are known. In general, known inhibitors of MMPs are chemically modified tetracyclines (CMTs), a number of which are listed below. The CMTs include, but are not limited to, 4-dimethylamino-TC (also known as CMT-1); tetracycinonitrile (CMT-2); 6-demethyl, 6-deoxy, 4-dedimethylamino-TC (CMT-3); 7-chloro, 4-dedimethylamino-TC (CMT-4); 4-hydroxy, 4-dedimethylamino-TC (CMT-6); 12a-deoxy, 5-hydroxy-4-dedimethylamino-TC (CMT-7); 6a-deoxy, 5 hydroxy-4-dedimethylamino-TC (CMT-8); 12a, 4a-anhydro; 4-dedimethylamino-TC (CMT-9); 7-dimethylamino, 4-dedimethylamino-TC (CMT-10). In addition to the CMTs, other known inhibitors of MMPs include the tissue inhibitors of MPs-1 and MPs-2 (TIMP-1 and TIMP-2, respectively) and minocycline (Min) and doxycycline (Dox). Suitable targeting moieties comprising peptide substrates for MMPs include the peptide sequence Pro-Met-Ala-Leu-Trp-Met-Arg (Netzel-Arnett, S., et al., 1993, Biochem., 32: 6427-6432). Recognition of the peptide sequence by an MMP can result in cleavage of the peptide sequence Pro-Met-Ala-Leu-Trp-Met-Arg to yield two peptide fragments: -Pro-Met-Ala- and -Leu-Trp-Met-Arg. Preferred peptide substrates include -Ala-Leu-. There are a number of other MMP inhibitors and substrates that can be used as targeting moieties. The substrates are particularly useful as cancer cleavage sites with the use of coordination site barriers. These MMP inhibitors and substrates include, but are not limited to, 1,10-phenanthroline; CT 1847; AG3319, AG3340 (also called Prinomastat), AG3287, AG3293, AG3294, AG3296; 2-mercaptoacetyl L-phenyl-alanyl-L-leucine; HSCH2 CH[CH2CH(CH3)2]CO-Phe-Ala-NH2; OPB-3206; Furin Inhibitor; 3,4-dihydro-1-oxo-1,2,3,-benzotriazine-3-(3-tetrahydrofuranyl)carbonate (IW-1); 1,2-dihydro-3,6dioxo-2-phenyl-pyridazine-1-methylcarbonate (LW-2); 3,4-dihydro-1-oxo-1,2,3,-benzotriazine-3-(2methoxy) ethylcarbonate (LW-3); 1,2-dihydro-2-ethoxycarbonyl-(1-oxo-isochinolin-5-yl) ethylcarbonate (LW-4); 1(2H)-phtalazinone-2-(4-methoxyphenyl) carbonate (LW-5); N-[2(R)-2-(hydroxamido carbonylmethyl)-4-methyl pentanoyl]-L-tryptophane methylamide also called GM6001, Galardin and ilomastat; BAY 12-9566; Neovastat (AE-941); BB-1101; G1129471; Ph(CH2NH-D-RrevCO-CH2CH2-D)2 also called FC-336; Mca-Pro-Leu-Gly-Leu-Dpa-Ala-Arg-NH2 (cleavage occurs between Gly and Leu); DNP-Pro-Leu-Gly-Ile-Ala-Gly-Arg-OOOH (cleavage occurs between Gly and Leu); arboxymethyl transferrin (Cm-Tf); (7-methoxycoumarin-4-yl)acetyl-PLGP-[3-(2,4-dinitrophenyl)-L-2,3 diaminopropionyl]-AR-NH2; (7-methoxycoumarin-4-yl)acetyl-PLAQAV-[3-(2,4-dinitrophenyl)-L-2,3 diaminopropionyl]-RSSSR-NH2; Ac-PLG-[2-mercapto-4-methylpentanoyl]-LG-OEt; Peptide I: GPLGLRSW; and Peptide II: GPLPLRSW. See generally, Greenwald, R. A. et al. In vitro sensitivity of the three mammalian collagenases to tetracycline inhibition: relationship to bone and cartilage degradation. Bone 22, 33-38 (1998); Kolb, S. A. et al. Matrix metalloproteinases and tissue inhibitors of metalloproteinases in viral meningitis: upregulation of MMP-9 and TIMP-1 in cerebrospinal fluid. J. Neuroimmunol. 84, 143-150 (1998); Charoenrat, P. et al. Overexpression of epidermal growth factor receptor in human head and neck squamous carcinoma cell lines correlates with matrix metalloproteinase-9 expression and in vitro invasion. Int. J. Cancer 86, 307-317 (2000); Uzui, H., Lee, J. D., Shimizu, H., Tsutani, H. & Ueda, T. The role of protein-tyrosine phosphorylation and gelatinase production in the migration and proliferation of smooth muscle cells. Atherosclerosis 149, 51-59 (2000); Montesano, R., Soriano, J. V., Hosseini, G., Pepper, M. S. & Schramek, H. Constitutively active mitogen-activated protein kinase kinase MEK1 disrupts morphogenesis and induces an invasive phenotype in Madin-Darby canine kidney epithelial cells. Cell Growth Differ. 10, 317-332 (1999); Yip, D., Ahmad, A., Karapetis, C. S., Hawkins, C. A. & Harper, P. G. Matrix metalloproteinase inhibitors: applications in oncology. Invest New Drugs 17, 387-399 (1999); Price, A. et al. Marked inhibition of tumor growth in a malignant glioma tumor model by a novel synthetic matrix metalloproteinase inhibitor AG3340. Clin. Cancer Res. 5, 845-854 (1999); Santos, O., McDermott, C. D., Daniels, R. G. & Appelt, K. Rodent pharmacokinetic and anti-tumor efficacy studies with a series of synthetic inhibitors of matrix metalloproteinases. Clin. Exp. Metastasis 15, 499-508 (1997); Barletta, J. P. et al. Inhibition of pseudomonal ulceration in rabbit corneas by a synthetic matrix metalloproteinase inhibitor. Invest Ophthalmol. Vis. Sci. 37, 20-28 (1996); Maquoi, E. et al. Inhibition of matrix metalloproteinase 2 maturation and HT1080 invasiveness by a synthetic furin inhibitor. FEBS Lett. 424, 262-266 (1998); Makela, M. et al. Matrix metalloproteinase 2 (gelatinase A) is related to migration of keratinocytes. Exp. Cell Res. 251, 67-78 (1999); Hao, J. L. et al. Effect of galardin on collagen degradation by Pseudomonas aeruginosa. Exp. Eye Res. 69, 595-601 (1999); Hao, J. L. et al. Galardin inhibits collagen degradation by rabbit keratocytes by inhibiting the activation of pro-matrix metalloproteinases. Exp. Eye Res. 68, 565-572 (1999); Wallace, G. R. et al. The matrix metalloproteinase inhibitor BB-1101 prevents experimental autoimmune uveoretinitis (EAU). Clin. Exp. Immunol. 118, 364-370 (1999); Maquoi, E. et al. Membrane type 1 matrix metalloproteinase-associated degradation of tissue inhibitor of metalloproteinase 2 in human tumor cell lines: J. Biol. Chem. 275, 11368-11378 (2000); Ikeda, T. et al. Anti-invasive activity of synthetic serine protease inhibitors and its combined effect with a matrix metalloproteinase inhibitor. Anticancer Res. 18, 4259-4265 (1998); Schultz, S. et al. Treatment of alkali-injured rabbit corneas with a synthetic inhibitor of matrix metalloproteinases. Invest Ophthalmol. Vis. Sci. 33, 3325-3331 (1992); Buchardt, J. et al. Phosphinic Peptide Matrix Metalloproteinase-9 Inhibitors by Solid-Phase Synthesis Using a Building Block Approach. Chem. Eur. J. 5, 2877-2884 (2000); Dahlberg, L. et al. Selective enhancement of collagenase-mediated cleavage of resident type II collagen in cultured osteoarthritic cartilage and arrest with a synthetic inhibitor that spares collagenase 1 (matrix metalloproteinase 1). Arthritis Rheum. 43, 673-682 (2000); Lombard, M. A. et al. Synthetic matrix metalloproteinase inhibitors and tissue inhibitor of metalloproteinase (TIMP)-2, but not TIMP-1, inhibit shedding of tumor necrosis factor-alpha receptors in a human colon adenocarcinoma (Colo 205) cell line. Cancer Res. 58, 4001-4007 (1998); Lein, M. et al. Synthetic inhibitor of matrix metalloproteinases (batimastat) reduces prostate cancer growth in an orthotopic rat model. Prostate 43, 77-82 (2000); Brown, P. D. Matrix metalloproteinase inhibitors in the treatment of cancer. Med. Oncol. 14, 1-10 (1997); Garbett, E. A., Reed, M. W. & Brown, N. J. Proteolysis in colorectal cancer. Mol. Pathol. 52, 140-145 (1999); Itoh, M. et al. Purification and refolding of recombinant human proMMP-7 (pro-matrilysin) expressed in Escherichia coli and its characterization. J. Biochem. (Tokyo) 119, 667673 (1996); Wang, Y., Johnson, A. R., Ye, Q. Z. & Dyer, R. D. Catalytic activities and substrate specificity of the human membrane type 4 matrix metalloproteinase catalytic domain. J. Biol. Chem. 274, 3304333049 (1999); Ohkubo, S. et al. Identification of substrate sequences for membrane type-1 matrix metalloproteinase using bacteriophage peptide display library. Biochem. Biophys. Res. Commun. 266, 308-313 (1999), all of which are expressly incorporated by reference, and all of which can be used as targeting moieties.

In a preferred embodiment, the targeting moiety is a substrate or inhibitor for matrilysin (also sometimes referred to in the literature as pump-1 and MMP-7). It has been implicated in gastric, colon, breast and prostate cancers, and is clearly implicated in metastasis and potentially growth and invasion as well. It is a zinc metalloenzyme, with a thermolysin-type Zn binding region, and is activated by cystein switch. It is exclusively associated with tumor cells, unlike other MMPs, and its mRNA expression is induced by IL-1. It is secreted from epithelial cells of glandular tissue. Its substrates include, but are not limited to, proteglycans, laminin, fibronectin, gelatins, collagen IV, elastin, entactin and tenascin. Its inhibitors include a variety of metal chelators and tissue inhibitors (TIMPs). See MacDougall et al., Cancer and Metastasis Rev. 14:351 (1995); Stetler-Stevenson et al., FASEB 7:1434 (1993); Mirelle Gaire et al., J. Biol. Chem. 269:2032 (1994), all of which are expressly incorporated by reference, and all of which can be used as targeting moieties.

In a preferred embodiment, the targeting moiety is a substrate or inhibitor for the extracellular statum corneum chymotryptic enzyme (SCCE), which has been implicated in ovarian cancer. This enzyme is involved in tumor invasion and metastasis by allowing implantation and invasion of neighboring cells. It is a serine protease with a standard catalytic triad (ser-his-asp) in its active site, and it may activate MMPs. Its substrates include gelatin and collagen, and is inhibited by the D43 mAb. See Tantimoto et al., supra; Hansson et al., J. Biol. Com. 269:19420 (1994), both of which are incorporated by reference, and all of which can be used as targeting moieties.

In a preferred embodiment, the targeting moiety is a substrate or inhibitor for seprase. Seprase has been implicated in breast cancer and is involved in an early event in the progression from a non-invasive premalignant phenotype to the invasive malignant phenotype. It is a 170 kDa dimer, and is a serine integral membrane protease (with a putative standard catalytic triad) with gelanitinase activity. The monomer 97 kDa form is inactive. The catalytic domain is exposed to the extracellular environment. Seprase is overexpressed in neoplasic invasive ductal carcinoma (IDC) cells and exhibits low levels of expression in benign proliferative tissue or normal breast cells. It also may activate MMPs. It degrades gelatin and collagen. See Kelly et al, Mod. Path. 11(9):855 (1998), incorporated by reference.

In a preferred embodiment, the targeting moiety is a substrate or inhibitor for Type IV collegenase (also sometimes referred to as MMP-2 and gelantinase A). This enzyme has been implicated in breast, colon and gastic cancers, and is involved in the penetration of membrane material and the invasion of stroma. It is a 72 kDa neutral Zn metalloendoproteinase that degrades basement membrane type IV collagen and gelatin in a pepsin-resistant domain. It is activated by a cysteine switch and is a membrane type I MMP. It is secreted extracellularly by epithelial cells, fibroblasts, endothelial cells and macrophages as an inactivated form. Its substrates include, but are not limited to, type IV collagen, gelatins, fibroblasts, type V collagens, type VII collagen, proMMP-9 and elastins. It's inhibitors include TIMP-2. See Poulsom et al., Am. J. Path. 141:389 (1992); Stearns et al., Cancer Res. 53:878 (1993); Nakahara et al., PNAS USA 94:7959 (1997); and Johnson et al., Curr. Opin. Chem. Biol. 2:466 (1999), all of which are expressly incorporated by reference, and all of which can be used as targeting moieties.

In a preferred embodiment, the targeting moiety is a substrate or inhibitor of HER-2/neu protein (sometimes referred to as erb-B-2). HER-2/neu is a 185 kDa transmembrane phosphoglycoprotein with tyrosine kinase activity that has been implicated in breast, ovarian and non-small cell (NSC) lung carcinoma. High serum levels have been shown to correlate with poor prognosis and increased resistance to endocrine therapy, and it has been identified in 25-30% of all breast cancers. Its ligands are NDF/heregulins and gp 30 (which is related to TGFa. See Codony-Serat et al., Cancer Res. 59:1196 (1999); Earp et al., Breast Canc. Res. Treat. 35:115 (1995); Depowski et al., Am. J. Clin. Pathol. 112:459 (1999), all of which are expressly incorporated by reference, and all of which can be used as targeting moieties.

In a preferred embodiment, the targeting moiety binds and/or inhibits ras, which has been implicated in NSC lung cancer. Ras is an essential signal transduction protein though to follow overexpression of HER2/neu protein, and is also related to p53 overexpression. Deregulated expression of ras results in uncontrolled cell growth and cancer, with overexpression being correlated with drug resistance. It functions as a surface antigen that is recognized by antibodies and T-cells. See Shackney et al., J. Thorac. Cadio. Surg 118:259 (1999), incorporated by reference, and all of which can be used as targeting moieties.

In a preferred embodiment, the targeting moiety binds to RCAS1. RCAS1 has been implicated in uterine, ovarian, esophageal and small cell lung carcinomas, gastic colon, lung and pancreatic cancers. It is a type 11 membrane protein and acts as aligand for a receptor on normal peripheral lymphocytes (e.g. T and NK cells) followed by inhibition of the receptor cell and cell death. It neutralizes immunoprotection by lymphocytes. It is expressed on cancer cell surfaces and in the extracellular medium, but is not detected in normal cells. See Nakashima et al., Nature Med. 5:938 (1999) and Villunger et al., Nature Medicine 5:874 (1999), incorporated by reference.

In a preferred embodiment, the targeting moiety binds to reg protein (including reg la and reglβ and pap). Reg has been implicated in pancreatic cancer, colorectal and liver carcinomas, and is present in acinar cell carcinoma, pancreatoblastoma, solid and cystic tumors and ductal cell carcinoma. See Rechreche et al., Int. J. Cancer 81:688 (1999) and Kimura et al., Cancer 70:1857 (1992), incorporated by reference.

In a preferred embodiment, the targeting moiety binds to thrombospondin-1, which has been implicated in pancreatic adenocarcinoma. It activates TGF-β, which is a key fibrogenic factor resulting in desmoplasia. See Cramer et al, Gastrent. 166 (4 pt 2):pA1116 (G4840) (1999); incorporated by reference.

In a preferred embodiment, the targeting moiety is a substrate or inhibitor for a caspase enzyme, including caspase-1 (also sometimes referred to as IL-1β), -3, -8, -9, etc. Caspases are also cysteine proteases which are putatively involved in the apoptosis cascade. Many of the caspases are generally made as proenzymes of 30-50 kDa. They cleave after asp residues with recognition of 4 amino acids on the N-side of the cleavage site.

In a preferred embodiment, the targeting moiety binds to alpha 1-acid glycoprotein (MG). MG has been suggested as a prognostic aid for glioma and metastatic breast and other carcinomas. MG is highly soluble and is a single 183 amino acid polypeptide chain. It is characterized by a high carbohydrate (45%) and sialic acid (12%) content, and a low isoelectric point (pH 2.7). It has been implicated in binding of many drugs, including propranolol, imipramine and chloropromazine, all of which can be used as a guarding moiety.

In a preferred embodiment, the targeting moiety is involved in angiogenesis. There are a wide variety of moieties known to be involved in angiogenesis, including, but not limited to, vascular endothelial growth factors (VEGF; including VEGF-A, VEGF-B, VEGF-C and VEGF-D), FGF-1 (aFGF), FGF-2 (bFGF), FGF-3, FGF-4, hepatocyte growth factor (HGF, scatter factor), thymidine phosphorylase, angiogenin, IL-8, TNF-a, leptin, transforming growth factors (TGF-a, TGF-β), platelet-derived growth factor, proliferin, and granulocyte colony stimulating factor (G-CSF). Known angiogenesis inhibitors include, but are not limited to, platelet factor 4, thrombospondin-1, interferons (IFN-a, IFN-β, IFN-?), IL-1, IL-2, vascular endothelial growth inhibitor (VEGI), 2-methoxyestradiol, tissue inhibitors of MMPs (TIMPs), proliferin related protein, angiostatin, endostatin, amion terminal fragment of u-PA (ATF), thalidomide, TNP-470/AGM-1470, carboxyamidotriazole, maspin, AG3340, marimastat, BAY9566, CSG-27023A, gly-arg-gly-asp-ser (GRGDS), tyr-ile-gly-ser-arg (YIGSR) and ser-ile-lys-val-ala-val (SIKVAV). See van Hinsbergh et al, Annals of Oncology 10 Supp. 4:60 (1999) and references therein; Li et al., Human Gene Therapy 10(18):3045 (1999); Duenas et al., Investigative Ophthalmology, 1999; Bauer et al., J. Pharmacology & Experimental Therapeutics 292(1):31 (2000); Zhang et al., Nature Medicine 6(2):196 (2000); Sipose et al., Annal of the New York Academy of Sciences 732:263 (1994 and references therein); Niresia et al, Am. J. Pathology 138(4):829 (1991); Yamamura et al., Seminars in Cancer Biology 4(4):259 (1993). Thus moieties which bind to these factors are useful as targeting moieties in the present invention.

In some embodiments, the targeting moiety is an antibody. The term “antibody” includes antibody fragments, as are known in the art, including Fab Fab2, single chain antibodies (Fv for example), chimeric antibodies, etc., either produced by the modification of whole antibodies or those synthesized de novo using recombinant DNA technologies or other technologies.

In some embodiments, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323-327 (1988); Verhoeyen et al., Science 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol. 227:381 (1991); Marks et al., J. Mol. Biol. 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology, 14:826 (1996); Lonberg and Huszar, Intern. Rev. Immunol. 13:65-93 (1995).

Bispecific antibodies are monoclonal, preferably human or humanized, antibodies that have binding specificities for at least two different antigens. In the present case, one of the binding specificities is for a first target molecule and the other one is for a second target molecule.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

Heteroconjugate antibodies are also within the scope of the present invention. Hetero-conjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a preferred embodiment, the antibody is directed against a cell-surface marker on a cancer cell; that is, the target molecule is a cell surface molecule. As is known in the art, there are a wide variety of antibodies known to be differentially expressed on tumor cells, including, but not limited to, HER2.

In addition, antibodies against physiologically relevant carbohydrates may be used, including, but not limited to, antibodies against markers for breast cancer (CA15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242). A particularly preferred carbohydrate targeting moiety will bind to enzyme β-glucuronidase, as outlined above.

In a preferred embodiment, the targeting moiety is a carbohydrate. By “carbohydrate” herein is meant a compound with the general formula Cx(H2O)y. Monosaccharides, disaccharides, and oligo- or polysaccharides are all included within the definition and comprise polymers of various sugar molecules linked via glycosidic linkages. Particularly preferred carbohydrates are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates. In particular, polysaccharides (including, but not limited to, arabinogalactan, gum arabic, mannan, etc.) have been used to deliver MRI agents into cells; see U.S. Pat. No. 5,554,386, hereby incorporated by reference in its entirety and can be used for the present triad compositions as well.

In addition, the use of carbohydrate targeting moieties can allow differential uptake into different tissues or altered half-life of the compound.

In a preferred embodiment, the targeting moiety is a lipid. “Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

In a preferred embodiment, the targeting moiety may be used to either allow the internalization of the triad agent to the cell cytoplasm or localize it to a particular cellular compartment, such as the nucleus.

In a preferred embodiment, the targeting moiety is all or a portion of the HIV? 1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); Baldin et al., EMBO J. 9:1511 (1990); Watson et al., Biochem. Pharmcol. 58:1521 (1999), all of which are incorporated by reference.

In a preferred embodiment, the targeting moiety is a nuclear localization signal (NLS). NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-1 nuclear localization signal (ARRRRP); NF?B p50 (EEVQRKRQKL; Ghosh et al., Cell 62:1019 (1990); NF?B p65 (EEKRKRTYE; Nolan et al., Cell 64:961 (1991); and others (see for example Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458,1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799,1987; Galileo, et al., Proc. Natl. Acad. Sci. USA, 87:458-462, 1990.

In a preferred embodiment, targeting moieties for the hepatobiliary system are used; see U.S. Pat. Nos. 5,573,752 and 5,582,814, both of which are hereby incorporated by reference in their entirety.

In addition to the targeting and imaging moieties, a PDT treatment moiety is included in the multifunctional agents of the invention. Photodynamnic therapy is an accepted treatment of tumors, as well as age related macular degeneration. PDT is initiated by introducing a photosensitizer agent into a subject's blood stream. After an appropriate time interval (usually tens of hours, which allows the accumulation of the agent at the appropriate site), the photosensitizer is activated by shining a visible light, usually a red color laser beam, at the donor's location. It should be noted that in the case of targeted agents such as described herein, the period of time for accumulation may be shortened, allowing shorter treatment times.

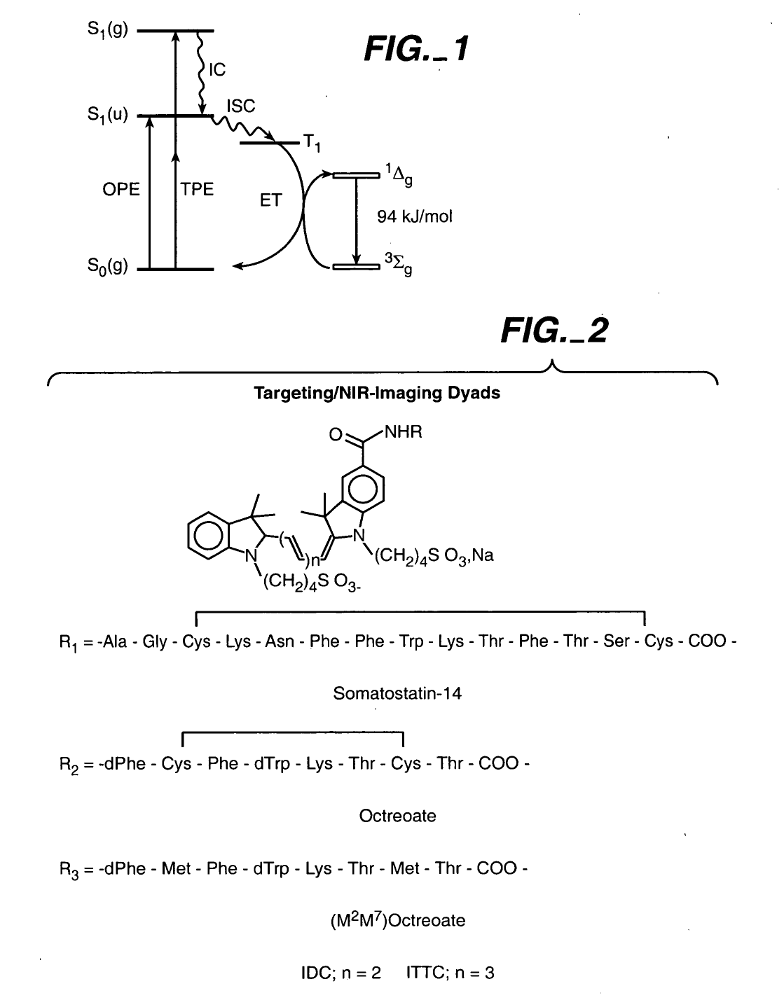

PDT employs the special ability of some porphyrin and porphyrin-like photosensitizers to accumulate in pathologic cells, and to transfer, upon or subsequent to radiation, absorbed photon energy to naturally occurring oxygen molecules in blood and tissue. Photophysical processes constituting PDT using porphyrin agents are summarized in the energy level diagram shown in FIG. 1.

In its classical implementation, absorption of one photon of visible wavelength takes a photosensitizer molecule into a short-lived excited state, S1, with energy of 170-190 kJ mol”, which corresponds to an illumination wavelength of about 620 to 690 nm. Alter a few nanoseconds, the porphyrin converts into a triplet state, T1, by an intersystem crossing (TSC) mechanism with energy of 110-130 kJ mot-1 and a much longer lifetime, on the order of milliseconds. From this triplet state, energy is transferred to omnipresent oxygen molecules by switching them from a triplet ground state, 3Σg, into an excited singles state, lΔg, which has an excitation energy of 94 kJ mol-1. Once in the excited singlet state, the oxygen presents an extremely active species, which reacts chemically with the surrounding cell material and causes tumor apoptosis.

The use of longer wavelength, near-infrared light to cause absorption of two photons of longer wavelength light has been developed to treat breast and other cancers, See U.S. Pat. Nos. 5,829,448, 5,832,931, 5,998,597, and 6,042,603. This two-photon technique employs a mode-locked Ti:sapphire laser to administer PDT with near-infrared light. In contrast to one-photon PDT, the near-infrared light produced by the Ti:sapphire laser is at a wavelength substantially longer than the characteristic one-photon absorption waveband of the photoreactive agent employed. Instead of the single photon absorption process involved in a conventional photodynamic reaction, a two photon process may occur upon radiation with a pulse of the 700-1300 nm light. Due to its relatively long wavelength, the near-infrared light emitted by a Ti:sapphire laser can penetrate into tissue up to 8 centimeter or more, making it possible to treat tumors that are relatively deep within a subject's body, well below the dermal layer. In addition, the use of endoscopes that are adapted to emit/receive light in the appropriate regions can be used for other types of deeper tissues, as will be appreciated by those in the art.

For photosensitizer molecules to be particularly efficacious they should selectively accumulate in the tumor tissue. It is known that porphyrin-based molecules possess this feature. To date, the U.S. Food and Drug Administration has approved at least two porphyrin-based PDT agents: Photofrin®, and Verteporfrin®. Photofrin® is a naturally occurring porphyrin, which absorbs light in the visible spectral range (X<690nn). However, neither of these compounds have significant absorption spectra in the near-infrared region of radiation of 700 to 1300 nm, nor do they exhibit efficient multi-photon absorption.

However, in some cases, a single photon PDT agent can be coupled to a targeting moiety to increase the specificity of the agent to accumulate at the desired location, and coupled to an imaging agent, as described below, to form trifunctional agents. Preferred embodiments utilize two photon PDT agents as either bifunctional agents with a targeting moiety or trifunctional agents with the addition of a imaging agent.

Chemical modification of the porphyrin structure, such as to chlorin or bacteriochlorin, to shift the one-photon absorption band to longer wavelengths is limited by the fundamental requirement that the energy of the TI state be higher than the excitation energy of singlet oxygen. Furthermore, such structural modification of the porphyrin structure may result in a less stable compound.

Non-porphyrin-based materials may have enhanced TPA cross-sections but typically lack either the ability to generate singlet oxygen, or have either unknown or deleterious interaction properties with biological tissue.

At the current time FDA approved PDT therapeutic agents only allow a few types of skin, metastatic breast and certain endoscopically accessible cancers to be treated, due to the lack of penetration of the light through the skin and surface tissue since the activation wavelength for these reagents is below 700 nm. To make PDT more generally applicable, it is crucial to deliver light deeper into the tissue. This may be achieved by utilizing the nonlinear optical effect of two-photon absorption (TPA), in which case the illumination is carried out at NIR wavelengths where the tissue is much more transparent. However, the TPA of most known porphyrins has been notoriously inefficient, rendering the PDT treatment of deep tumors impractical. We have recently reported the synthesis of a new porphyrin sensitizer (see reference 16, incorporated by reference) with enhanced TPA cross-section, and have demonstrated its ability to generate singlet oxygen upon illumination with NIR light. The processes involved in TPA by the porphyrin, and formation of singlet oxygen, are shown in FIG. 1. Note that after TPA, intersystem crossing from the excited singled to a triplet state occurs, with subsequent formation of singlet oxygen dissolved in the solution (or in the blood for a tumor).

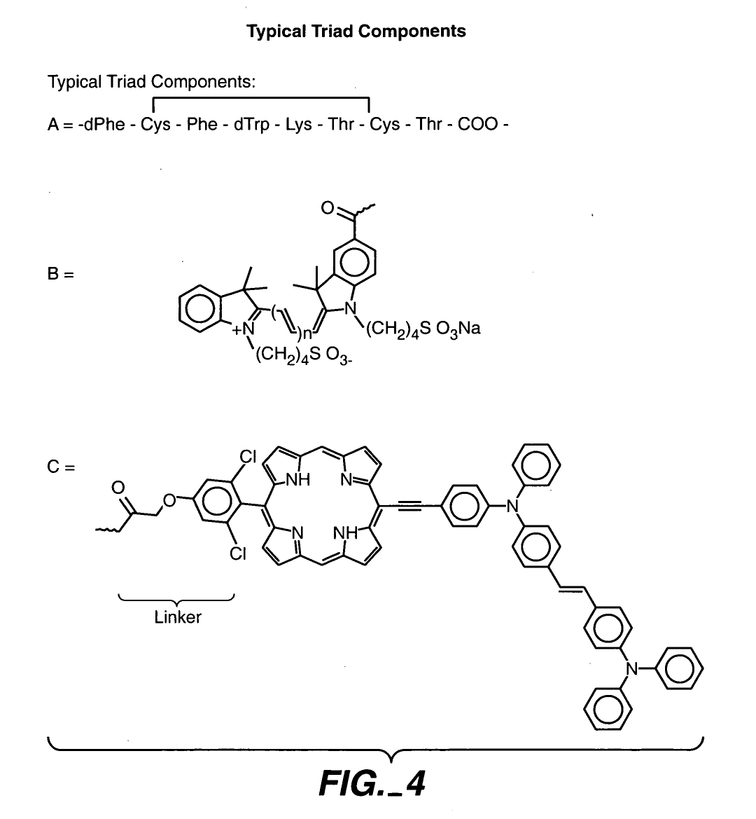

Porphyrins currently in use in FDA-approved photodynamic applications fall short of having their absorption in the tissue transparency window (800-1000 nm), since their S0 to S1 transition usually falls in the region 620-690 nm, where effective penetration through the skin is only a few millimeters. Unfortunately, attempts to shift the one-photon absorption band toward higher wavelength (red shift) by chemical modification of the porphyrin structure come in conflict with the fundamental requirement that the excitation energy of singlet oxygen be lower than the energy of the T1 state. In addition, long-wavelength shifts in the porphyrin's energy level often aggravate the situation by reducing the porphyrin's photostability. Both 1 and 2 photon PDT compounds, with the latter being preferred, find use in the present invention. In particular, those 2 photon moieties described in PCT US02/26626, filed 22 Aug. 2002, also U.S. Publication No. 2003/0105070, hereby incorporated by reference in its entirety, are preferred, particularly 2PM agents shown in the figures, and particularly porphyrin molecules modified with at least one TPA chromophore that result in the 2PM moieties. It should be noted that in general, structures within U.S. Publication No. 2003/0105070 can be used by attachment in any number of locations, as generally described below, with attachment to the linker (and thus the other components of the agents herein) using a carbon of the porphyrin ring being preferred. As shown in FIG. 4, an additional linker may be used to attached to the core linker, as depicted in C. It should additionally be noted that the same TPA chromophore that is used to form the 2PM when coupled to a porphyrin may be used as an imaging agent. Alternatively, one TPA chromophore is used to form the 2PM with a porphyrin and another for the imaging moiety.

In addition to targeting moieties, preferred embodiments utilize an imaging moiety. There are a variety of suitable imaging moieties which may be used, including, but not limited to, optical imaging agents (including chromophores and fluorophores), as well as imaging agents based on other technologies such as MRI and PET contrast agents.

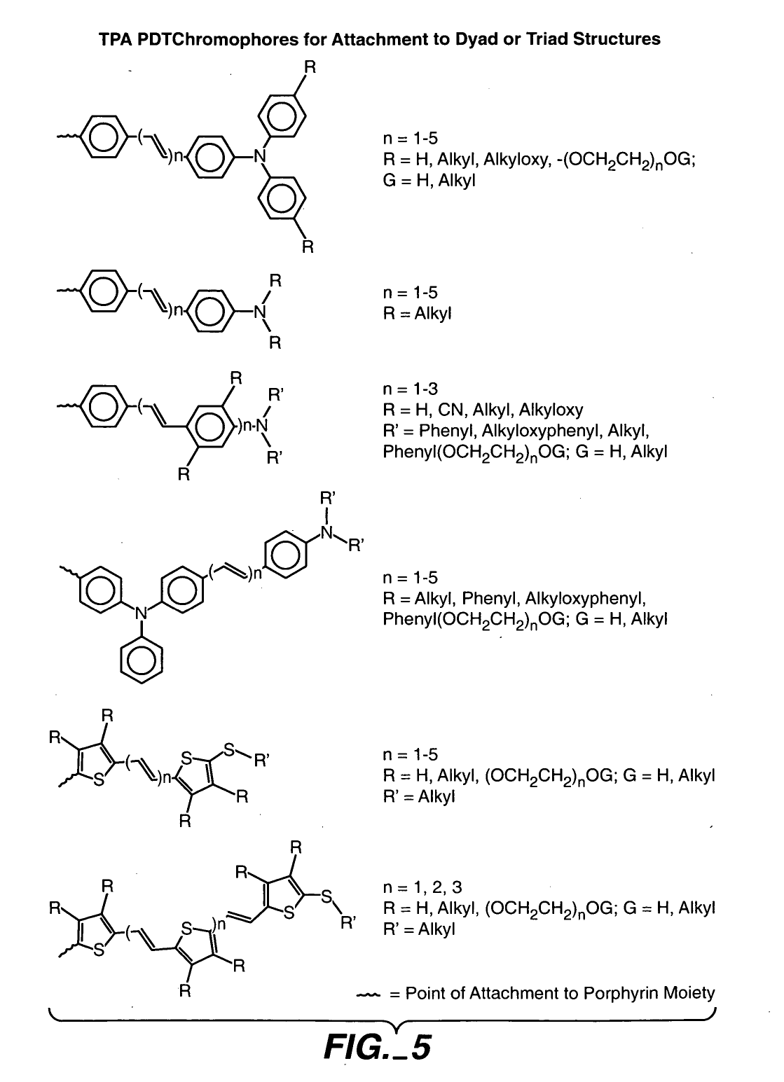

A preferred embodiment utilizes one photon chromophores, as are known in the art, some of which are shown in FIG. 5.

In addition to the methods outlined herein, the agents of the invention can be coupled with other imaging modalities. Evaluation of several of these technologies are, in fact, being funded by NIH (NCI) at the current time, including a $25 million study being conducted by Johns Hopkins Medicine Department of Radiology, funded by NCI, and named the American College of Radiology Imaging Network, which will examine 49,500 women in the U.S. and Canada to compare the relative merits of traditional and digital mammography. The following brief listing includes some of the more promising imaging technologies. It should again be emphasized that while the following discussion emphasizes breast cancer, the same arguments hold for other types of solid cancerous tumors and in some cases, many other disease states. All of these imaging modalities have effective agents that can be aduvants to the technoogy described in this application.

Digital Mammography—Compared to traditional mammography, digital mammography records X-ray data in computer code instead of on X-ray film. The procedure for a digital mammogram is the same as conventional mammography, and since images are stored electronically, long-distance consultations are possible. However, it has been reported (see reference 10, incorporated by reference) that studies have not yet conclusively shown that digital mammography is more effective in detecting cancer than traditional mammography.

Computer-Aided Detection—This technique involves the use of computers to bring suspicious areas already found by conventional mammography to a radiologist's attention. This is not a replacement technology, but rather an enhancement. CAD marks regions of the breast that a radiologist may wish to examine more closely. CAD technology for breast imaging was approved by the FDA in 1998, and R2 Technology, Inc. has marketed a detection system called ImageChecker, and sold ca. 200 units worldwide.

Magnetic Resonance Imaging (MRI)—Magnetic resonance imaging (see reference 10, incorporated by reference) is similar to the nuclear magnetic imaging systems used extensively to determine the structures of compounds, in that radio frequency radiation is utilized instead of X-rays. The process is very accurate in obtaining detailed pictures of soft tissue, but requires a long patient session (up to 1 hour) where the patient must remain still, and some machines are very claustrophobic. MRI cannot always distinguish between cancerous and benign tissue, and it can detect microcalcifications and possibly reduce the number of false positives. MRI contrast agents can produce images that are much clearer than those obtained from conventional mammography, and MRI signal are not compromised by signals from fat deposits. Siemens, Marconi, Phillips Medical Systems and GE all have systems under development, and NIH is sponsoring a consortium of 14 universities and research centers to evaluate MRI as a diagnostic tool for breast cancer. As noted above, MRI contrast agents such as DOTA and DTPA derivatives can be used as imaging agents, or MRI (with or without contrast) can be used as an adjuvant imaging step.

To date, a number of chelators for the paramagnetic ions that form the basis of the contrast in MRI have been used, including diethylenetriaminepentaacetic (DTPA), 1,4,7,10-tetraazacyclododecane′-N,N′N″,N′″-tetracetic acid (DOTA), and derivatives thereof. See U.S. Pat. Nos. 5,155,215, 5,087,440, 5,219,553, 5,188,816, 4,885,363, 5,358,704, 5,262,532, and Meyer et al., Invest. Radiol. 25: S53 (1990).

Ultrasound (Sonography)—Ultrasound imaging techniques bounce sound waves off of tissue and internal organs, and produce an echo picture called a sonogram. Ultrasound can be used to evaluate lumps in the breast that are difficult to see in a mammogram, and can distinguish between solid tumors and fluid-filled cysts. 3D ultrasound techniques (see reference 12) can detect abnormal blood vessel activity in the breast associated with tumors, and can image to depths of 2 inches. Ultrasound does not consistently detect early signs of cancer.

Positron Emission Tomography (PET). PET scans create computerized images of chemical changes in tissue by injecting a patient with a low dose of a radioactive tracer. After ingesting the tracer, the patient must lie still for ca. 45 minutes, after which the PET scanner takes images for an additional 45 minutes and quantifies the position and concentration of the radionuclide to produce high-resolution images. PET scans are very accurate in detecting large and more aggressive tumors, but are not good at detecting tumors smaller than 8 mm, or ones that are not aggressive. PET tracers can be used as imaging moieties in the present invention, or a PET scan is used as an adjuvant to the methods of imaging of the present invention.

Electrical Impedance Scanning. EIS measures the speed that electricity travels through materials. Breast cancer tissue has a much lower electrical impedance than does normal tissue. These devices are used in combination with traditional mammography, and can detect abnormal areas not detected by the mammography. It is not approved or utilized as a stand-alone screening device for breast cancer.

Optical Coherence Tomography (OCT). OCT is similar to ultrasound in that both create images by bouncing waves off tissue, but using light rather than sound. It does not require a conducting medium and therefore can image through water and air. The technique uses two NIR beams to create interference patterns that can be translated into two- and three-dimensional high resolution images. Advanced Research Technologies, Inc. has a system called SoftScan in clinical trials in which the optical images will be compared to traditional mammography and biopsies. Researchers at the Beckman Laser Institute (U. Cal.-Irvine) (B. Tromberg) have developed a laser-based breast tissue scanner that can capture a complete spectral picture from 600-1000 nm in ca. 30 seconds to depths of centimeters with no breast compression (see reference 13, incorporated by reference). The technique quantifies the concentration of oxygenated and deoxygenated hemoglobin, water and fat, as well as total hemoglobin content. The scanner, comprised of 10 NIR lasers and a broad band light source to shine through breast tissue, separates the effects of absorption and scattering by modulating the laser light source at frequencies ranging from MHz to GHz, creating a diffuse photon density wave that travel through the tissue with a given phase velocity. In initial studies, the scanner was able to detect normal changes in breast tissue associated with age differences, varying tissue densities and hormone levels. Comparisons to conventional mammography and biopsies are planned. A similar approach at Clemson University (H. Jiang) has been able to detect carcinomas smaller than 5 mm using 785 nm light through 16-3 mm fiberoptic bundles (see reference 14, incorporated by reference). Researchers at Dartmouth College (T. McBride) have obtained similar results with 16-3 mm fiber bundles and a Ti:sapphire laser operating in the region 600-1100 nm (see reference 15, incorporated by reference). Optical tomography has been shown to be capable of detecting and characterizing sub-centimeter objects embedded within a 10 cm diameter region.

Imaging Diagnostic Systems, Inc. (Plantation, Fla.) is developing a system called Computed Tomography Laser Mammography (CTLM) that is currently being evaluated by the FDA, and is being marketed in Europe. In the U.S., CTLM systems have been installed at the Women's Center of Radiology (Orlando, Fla.), the Elizabeth Wende Breast Clinic (Rochester, N.Y.) and FDA approval to place a total of 10 CTLM systems in the U.S. under the IDE program has been obtained. The system utilizes state-of-the-art laser technology and proprietary algorithms to create contiguous cross-sectional images of the breast (every 4 mm) without the use of breast compression. They have also developed phantoms with optical properties similar to breast tissue to aid in the development of the CTLM system. Localization of NIR fluorophores as markers has been successfully demonstrated in the phantoms. This system produces 3-D projections of the breast that can be viewed from any angle, and a complete image can be obtained in 15-20 minutes while the patient lies prone on the scanning bed.

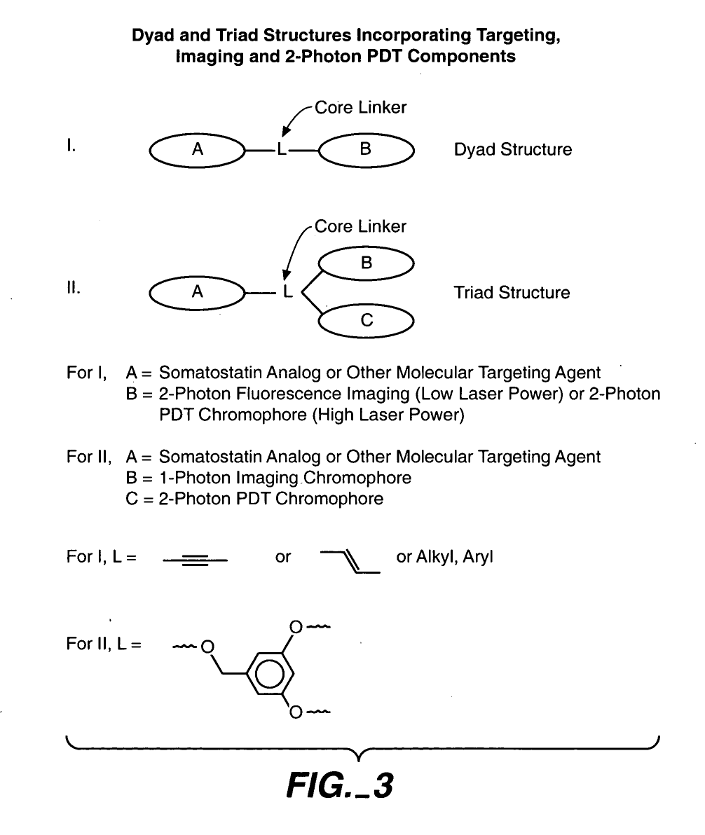

Thus, a preferred embodiment is shown in FIGS. 2 and 3, which depict dyads (bifunctional agents), comprising any or all of: (1) a one photon PDT moiety with a targeting moiety (shown in the figure as somatostain-14, octreoate or a derivative, but any of the above targeting moieties are included, with peptides being particularly preferred); (2), a two photon PDT moiety (2PM) with a targeting moiety; (3) a one photon PDT moiety, a targeting moiety and an imaging moiety; or (4) a two photon PDT moiety, a targeting moiety and an imaging moiety.

Generally, the three components of the triad composition are covalently attached. This can be accomplished in a number of ways. The synthesis of the A and B components illustrated in FIG. 1, and their combination as an indotricarbocyanine-peptide conjugate have already been described. Becker, A., Hessenius, C., Licha, K., et al. “Receptor-targeted Optical Imaging of Tumors with Newar-infrared Fluorescent Ligands”. Nature Biotech. 19:327 (2001); Achilefu, A., Dorshow, R. B., Bugai, J. E., Rajagopalan, R. “Novel Receptor-targeted Fluorescent Contrast Agents for In Vivo Tumor Imaging”. Investig. Radiology 35:479 (2000), 36. Licha, K., Riefke, B., Ntziachristos, V., Becker, A., Chance, B., Semmler, W. “Hydrophilic Cyanine Dyes as Contrast Agents for Near-infrared Tumor Imaging: Synthesis, Photophysical Properties and Spectroscopic In Vivo Characterization”. Photochem. Photobiol. 72:392 (2000).

The somatostatin receptor-specific peptide is prepared via Fmoc solid state peptide synthesis, and in the last step the dye is usually attached through the N-terminus of the peptide, followed by cleavage from the resin. In our new triad ensemble, the one-photon NIR imaging agent (e.g. ITTC) and the two-photon PDT porphyrin can be combined as part of an AB2 dendron in a manner similar to Frechet dendrimer methodology (see reference 37, incorporated by reference), and then reacted with the N-terminus of the octreoate followed by cleavage from the resin. This approach is outlined in Scheme 1. Note that the modes of attachment and combination will allow any combination of targeting, imaging and PDT reagents, and thus this approach can be modified for any tumor type. Many other modes of linking the three components using standard organic syntheic procedures can be envisioned.

In one embodiment, the components are linked together directly, using at least one functional group on each component. In this embodiment, the components of the invention include one or more substitution groups that serve as functional groups for chemical attachment. Suitable functional groups include, but are not limited to, amines (preferably primary amines), carboxy groups, and thiols (including SPDP, alkyl and aryl halides, maleimides, a-haloacetyls, and pyridyl disulfides) are useful as functional groups that can allow attachment.

This may be accomplished using any number of stable bifunctional groups well known in the art, including homobifunctional and heterobifunctional linkers (see Pierce Catalog and Handbook, 1994, pages T155-T200, hereby expressly incorporated by reference). This may result in direct linkage, for example when one chelator comprises a primary amine as a functional group and the second comprises a carboxy group as the functional group, and carbodiimide is used as an agent to activate the carboxy for attach by the nucleophilic amine (see Torchilin et al., Critical Rev. Therapeutic Drug Carrier Systems, 7(4):275-308 (1991). Alternatively, as will be appreciated by those in the art, the use of some bifunctional linkers results in a short coupling moiety or linker being present in the structure. A “coupling moiety” or “linker” is capable of covalently linking two or more entities The functional group(s) of the coupling moiety are generally attached to additional atoms, such as alkyl or aryl groups (including hetero alkyl and aryl, and substituted derivatives), to form the coupling moiety. Oxo linkers are also preferred. As will be appreciated by those in the art, a wide range of coupling moieties are possible, and are generally only limited by the ability to synthesize the molecule and the reactivity of the functional group. Generally, the coupling moiety comprises at least one carbon atom, due to synthetic requirements; however, in some embodiments, the coupling moiety may comprise just the functional group.

In a preferred embodiment, the coupling moiety comprises additional atoms as a spacer. As will be appreciated by those in the art, a wide variety of groups may be used. For example, a coupling moiety may comprise an alkyl or aryl group substituted with one or more functional groups. Thus, in one embodiment, a coupling moiety containing a multiplicity of functional groups for attachment of multiple components may be used, similar to the polymer embodiment described below. For example, branched alkyl groups containing multiple functional groups may be desirable in some embodiments.

By “alkyl group” or grammatical equivalents herein is meant a straight or branched chain alkyl group, with straight chain alkyl groups being preferred. If branched, it may be branched at one or more positions, and unless specified, at any position. The alkyl group may range from about 1 to about 30 carbon atoms (C1 ? C30), with a preferred embodiment utilizing from about 1 to about 20 carbon atoms (C1 ? C20), with about C1 through about C12 to about C15 being preferred, and C1 to C5 being particularly preferred, although in some embodiments the alkyl group may be much larger. Also included within the definition of an alkyl group are cycloalkyl groups such as C5 and C6 rings, and heterocyclic rings with nitrogen, oxygen, sulfur or phosphorus. Alkyl also includes heteroalkyl, with heteroatoms of sulfur, oxygen, nitrogen, and silicone being preferred. Alkyl includes substituted alkyl groups. By “substituted alkyl group” herein is meant an alkyl group further comprising one or more substitution moieties “R”, as defined above.

By “aromatic group” or “aryl group” or grammatical equivalents herein is meant an aromatic monocyclic or polycyclic hydrocarbon moiety generally containing 5 to 14 carbon atoms (although larger polycyclic rings structures may be made) and any carbocylic ketone or thioketone derivative thereof, wherein the carbon atom with the free valence is a member of an aromatic ring. Aromatic groups include arylene groups and aromatic groups with more than two atoms removed. For the purposes of this application aromatic includes heterocycle. “Heterocycle” or “heteroaryl” means an aromatic group wherein 1 to 5 of the indicated carbon atoms are replaced by a heteroatom chosen from nitrogen, oxygen, sulfur, phosphorus, boron and silicon wherein the atom with the free valence is a member of an aromatic ring, and any heterocyclic ketone and thioketone derivative thereof. Thus, heterocycle includes thienyl, furyl, pyrrolyl, pyrimidinyl, oxalyl, indolyl, purinyl, quinolyl, isoquinolyl, thiazolyl, imidozyl, etc.

Suitable R groups include, but are not limited to, hydrogen, alkyl, alcohol, aromatic, amino, amido, nitro, ethers, esters, aldehydes, sulfonyl, silicon moieties, halogens, sulfur containing moieties, phosphorus containing moieties, and ethylene glycols. In the structures depicted herein, R is hydrogen when the position is unsubstituted. It should be noted that some positions may allow two substitution groups, R and R′, in which case the R and R′ groups may be either the same or different

In an additional embodiment, the linker is a polymer. In this embodiment, a polymer comprising at least one triad agent of the invention is used. The targeting moieties can be added to the individual triads, multimers of the triads, or to the polymer. Preferred embodiments utilize a plurality of triad agents per polymer. The number of triad agents per polymer will depend on the density of triad agents per unit length and the length of the polymer.

The character of the polymer will vary, but what is important is that the polymer either contain or can be modified to contain functional groups for the attachment of agents of the invention. Suitable polymers include, but are not limited to, functionalized dextrans, styrene polymers, polyethylene and derivatives, polyanions including, but not limited to, polymers of heparin, polygalacturonic acid, mucin, nucleic acids and their analogs including those with modified ribose-phosphate backbones, the polypeptides polyglutamate and polyaspartate, as well as carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, spermine, spermidine and polypeptides such as protamine, the histone polypeptides, polylysine, polyarginine and polyornithine; and mixtures and derivatives of these. Particularly preferred polycations are polylysine and spermidine, with the former being especially preferred. Both optical isomers of polylysine can be used. The D isomer has the advantage of having long-term resistance to cellular proteases. The L isomer has the advantage of being more rapidly cleared from the subject. As will be appreciated by those in the art, linear and branched polymers may be used. A preferred polymer comprising a poly(alkylene oxide is also described in U.S. Pat. No. 5,817,292, incorporated by reference.

A preferred polymer is polylysine, as the —NH2 groups of the lysine side chains at high pH serve as strong nucleophiles for multiple attachment of activated chelating agents.

The synthesis of the compound can be done as outlined herein and as is generally known in the art.

Once made, the triad compositions can be used in a variety of applications, and in general include the imaging and treatment of disease, including cancer, cardiovascular disease (e.g. plaques, etc.), and other related disorders. As noted herein, the agents may be bifunctional (containing a targeting moiety and a PDT moiety, preferably a chromophore or fluorophore, with a particularly preferred embodiment being a two photon chromophore), or trifunctional (containing a targeting moiety, an imaging moiety, and a PDT moiety, with preferred embodiments utilizing imaging moieties of one-photon chromophores or fluorophores being preferred, and two photon PDT chromophores being particularly preferred as PDT agents). In addition, the agents can be used in optical imaging systems, either external systems or internal (e.g. endoscopic) systems, and can be used by themselves (with the appropriate imaging modality), or in combination with other imaging modalities, such as digital mammography, EIS<OCT, MRI, PET, etc.

Thus, one aspect of the present invention provides pharmaceutically acceptable compositions which comprise a therapeutically-effective amount of the triad compositions, such as described above, formulated together with one or more pharmaceutically acceptable carriers (additives) and/or diluents. As described in detail below, the pharmaceutical compositions of the present invention may be specially formulated for administration in solid or liquid form, including those adapted for parenteral administration, for example, by subcutaneous, intramuscular or intravenous injection as, for example, a sterile solution or suspension.

The phrase “therapeutically-effective amount” as used herein means that amount of a triad compound according to the present invention which is effective for producing some desired therapeutic effect.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject antioxidant or antimycotic agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Certain embodiments of the present compositions may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of the compounds of the invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of a compound herein. These salts can likewise be prepared in situ during the final isolation and purification of the compound or by separately reacting derivatives comprising carboxylic or sulfonic groups with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the triad compound which produces a therapeutic effect. Generally, out of one hundred percent, this amount will range from about 0.1 percent to about 99.5 percent of active ingredient, preferably from about 5 percent to about 70 percent, most preferably from about 10 percent to about 30 percent.

Pharmaceutical compositions of this invention suitable for parenteral administration comprise one or more of the triad compositions in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsuled matrices of the subject peptides or peptidomimetics in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

### LITERATURE CITED

1. Goodbar, D. E. “Light and Death: Photons and Apoptosis”. J. Investigative Dermatology Symp. Proc., 11:17 (1999).

2. Oleinick, N. L., He, J., Xue, L., Separovic, D. “Stress-activated Signaling Responses Leading to Apoptosis Following Photodynamic Therapy”. Prog. Biomed. Optics, Proc. SPIE, 3247:82 (1998).

3. Kessel, D., Luo, Y., Kim, H.-R. C. “Determinants of PDT-Induced Apoptosis”. Prog. Biomed. Optics, Proc. SPIE, 3903:76 (2000).

4. Oleinick, N. L., Belichenko, I., Chiu, S., Lam, M. C., Morris, R. L., Xue, L. “PDT-induced Apoptosis: What are the Critical Molecular Targets”. Prog. Biomed. Optics, Proc. SPIE, 4248:132 (2001).

5. Fisher, W. G., Partridge, W. P., Dees, C., Wachter, E. “Simultaneous Two-Photon Activation of Type-1 Photodynamic Therapy Agents”. Photochem. Photobiol. 66:141 (1997).

6. Wachter, E. A., Partridge, W. P., Fisher, W. G., Dees, H. C., Peterse, M. G. “Simultaneous Two-Photon Excitation of Photodynamic Therapy Agents”. Proc. SPIE, 3269:68 (1998).

7. Bhawalkar, J. D., Kumar, N. D., Zhao, C.-F., Prasad, P. N. “Two-Photon Photodynamic Therapy”. J. Clin. Laser Medicine and Surgery 66:201 (1997).

8. Photofrin is a proprietary Porphyrin-based PDT reagent offered by QLT, Inc. Vancouver, B.C.

9. All data reporting cancer rates and mortalities were obtained from the NCI and ACS websites

10. NCI Cancer Facts “Improving Methods for Breast Cancer Detection and Diagnosis—Digital Mammography” (Jun. 12, 2001)

11. Reiss, S. M. “Tomorrow's MRI: It's Not Your Father's MRI”. Biophotonics Int., p. 50-55 Nov. (2000).

12. Rowell, N. D. “Biophotonics News: 3-D Ultrasound Makes Breast Cancer Detection Easier”. Biophotonics Int., p. 32-33 Nov. (2000).

13. Hogan, H. “Laser Imaging Beats Mamography for Detail”. Biophotonics News, p. 26-29 June (2000).

14. Rowell, N. D. “Laser Imaging Beats Mamography for Detail”. Biophotonics Int., p. 32 December (2000).

15. Robinson, K. “Biophotonics Research: Optical Tomography Images Breast Tissue”. Biophotonics Int., p. 48-49 March (2000).

16. Karotki, A., Kruck, M., Drobizhev, M., Rebane, A., Spangler, C. W. “Efficient Singlet Oxygen Generation Upon Two-photon Exitation of new Porphyrin with Enhanced Nnlinear Absorption”. IEEE J. Select. Top. Quant. Electronics 7:961 (2001).

17. Reubi, J. C. “Neuropeptide Receptors in Health and Disease: The Molecular Basis for In Vivo Imaging”. J. Nucl. Med. 36:1825 (1995).

18. Troy, T. L., Page, D. L., Sevick-Muraca, E. M. “Optical Properties of Normal and Diseased Breast Tissues: Prognosis for Optical Mammography”. J. Biomed. Opt. 1:342 (1996).

19. Heusmann, H., Kolzer, J., Mitic, G. “Characterization of Female Breasts In Vivo by Time resolved and Spectroscopic Measurements in Near Infrared Spectroscopy”. J. Biomed. Opt. 1:425 (1996).

20. Wagnieres, G. A., Star, W. M., Wilson, B. C. “In Vivo Fluorescence Spectroscopy and Imaging for Oncological Applications”. Photochem. Photobiol. 68:603 (1998).

21. Baum, R. P., Brummendorf, T. H. “Radioimmunolocalization of Primary and Metastatic Breast Cancer”. J. Nucl. Med. 42:33 (1998).

22. Ballou, B., Fisher, G. W., Waggoner, A. S., et al. “Tumor Labeling In Vivo Using Cyanine-conjugated Monoclonal Antibodies”. Cancer Immunol. Immunother. 41:257 (1995).

23. Folli, S., Westermann, P., Braichiotte, D., et al. “Antibody-indocyanin Conjugates for Immunodetection of Human Squamous Cell Carcinoma in Nude Mice”. Cancer Res. 54:2643 (1994).

24. Becker, A., Reifke, B., Ebert, B., et al. “Macromolecular Contrast Agents for Optical Imaging of Tumors: Comparison of Indotricarbocyanin-labeled Human Serum Albumin and Transferrin”. Photochem. Photobiol. 72:234 (2000).

25. Reubi, J. C. “Relevance of Somatostatin Receptors and Other Peptide Receptors in Pathology”. Endocr. Pathol. 8:11 (1997).

26. Reubi, J. C., Lang, W., Maurer, R., Koper, J. W., Lamberts, S. W. J. “Distribution and Biochemical Characterization of Somatostatin Receptors in Tumors of the Human Central Nervous System”. Cancer Res. 47:5758 (1987).

27. Schaer, J. C., Waser, B., Mengood, G., Reubi, J. R. “Somatostatin Receptor Subtytpes sst1, sst2, sst3, and sst5 Expression in Human Pituitary Gasteroentero, Pancreatic and Mammary Tumors: Comparison of mRNA Analysis with Receptor Autoradiography”. Int. J. Cancer 70:530 (1997).

28. Lamberts, S. W. J., Bakker, W. H., Reubi, J. C., Krenning, E. P. “Somatostatin Receptor Imaging. In vivo Localization of Tumors with a Radiolabeled Somatostatin Analog”. J. Steroid Biochem. Mol. Biol. 37:1079 (1990).

29. Becker, A., Hessenius, C., Licha, K., et al. “Receptor-targeted Optical Imaging of Tumors with Newar-infrared Fluorescent Ligands”. Nature Biotech. 19:327 (2001).

30. Denzler, B., Reubi, J. C. “Expression of Somatostatin Receptors in Peritumoral Veins of Human Cancer”. Cancer 85:188 (1999).

31. Reubi, J. C., Schaer, J. C., Wasser, B., Mengood, G. “Expression and Localization of Somatostatin Receptor SSTR1, SSTR2, and SSTR3 Messenger RNAs in Primary Human Tumors Using in situ Hybridization”. Cancer Res. 54:3455 (1994).

32. Reubi, J. C., Kappeler, A., Wasser, B., Laissure, J., Hipkin, R. W., Schonbrunn, A. “Immunohistochemical Localization of Somatostatin Receptors ss2A in Human Tumors”. Am. J. Pathol. 153:233 (1998).

33. Hawrysz, D. J., Sevick-Muraca, E. M. “Developments Toward Diagnostic Breast Cancer Imaging Using Near-infrared Optical Measurements and Fluorescent Contrast Agents”. Neoplasia 2:388 (2000).

34. Flanagan, J. H., Khan, S. H., Menchen, S., Soper, S. A., Hammer, R. P. “Functionalized Tricarbocyanine Dyes as Near-infrared Fluorescent Probes for Biomolecules”. Bioconjugate Chem. 8:751 (1997).

35. Achilefu, A., Dorshow, R. B., Bugai, J. E., Rajagopalan, R. “Novel Receptor-targeted Fluorescent Contrast Agents for In Vivo Tumor Imaging”. Investig. Radiology 35:479 (2000).

36. Licha, K., Riefke, B., Ntziachristos, V., Becker, A., Chance, B., Semmler, W. “Hydrophilic Cyanine Dyes as Contrast Agents for Near-infrared Tumor Imaging: Synthesis, Photophysical Properties and Spectroscopic In Vivo Characterization”. Photochem. Photobiol. 72:392 (2000).

37. Frechet, J. M. J., Tomalia, D. A. “Dendrimers and Other Dendritic Polymers”. Chichester: John Wiley & Sons, Ltd., (2001).

We claim:

1. A trifunctional agent comprising:
a) a targeting moiety;
b) a medical imaging agent; and
c) a photo dynamic therapy (PDT) moiety.

2. A trifunctional agent according to claim 1 further comprising a linker moiety.

3. A trifunctional agent according to claim 1 wherein said medical imaging agent is a chromophore.

4. A trifunctional agent according to claim 1 wherein said PDT moiety is a porphyrin.

5. A trifunctional agent according to claim 4 wherein said PDT moiety is a substituted porphyrin.

6. A trifunctional agent according to claim 5 wherein said substituted PDT moiety is a two photon absorption PDT agent.

7. A method of imaging and treating a cancer comprising administering the agent of claim 1 to patient and administering light sufficient to activate the PDT agent.
